(19) United States
(12) Patent Application Publication
(10) Pub. No.: US 2020/0229962 A1
(43) Pub. Date: Jul. 23, 2020

(54) DRAINABLE OSTOMY POUCH OUTLET

(71) Applicant: Hollister Incorporated, Libertyville, IL (US)

(72) Inventors: Jan Torstensen, Virum (DK); Robert J. Weinberg, Lake Villa, IL (US); Timothy A. Friske, Round Lake Beach, IL (US)

(21) Appl. No.: 16/486,666

(22) PCT Filed: Apr. 4, 2018
(86) PCT No.: PCT/US18/26019

§ 371 Date: Aug. 16, 2019

Related U.S. Application Data
(60) Provisional application No. 62/481,876, filed on Apr. 5, 2017.

(51) Int. Cl. A61F 5/445; A61F 5/44

(52) U.S. Cl. CPC A61F 5/445; A61F 5/4407

(57) ABSTRACT

A drainable ostomy pouch outlet includes a single-piece closure member including at least one transversely-extending fold-line. The at least one transversely-extending fold-lines facilitates folding up and closing of the outlet.

### BACKGROUND

The present disclosure generally relates to ostomy appliances, and more particularly to drainable ostomy pouches having closure systems.

Ostomy pouches for collecting body waste are used by patients who have had surgery such as a colostomy, ileostomy, or urostomy. Ostomy pouches typically include flat, opposing side walls secured together along their edges to define a collection cavity. One of the side walls is provided with an opening to receive a stoma, and means to secure the pouch to the user, such as an adhesive barrier, so that body waste discharged through the stoma is received within the cavity.

The ostomy pouch may be a closed-end pouch for a single use, in which case the entire pouch is discarded after it has been substantially filled with stomal discharge. Alternatively, the ostomy pouch may be a drainable pouch with a discharge opening at its lower end, which may be closed during collection of body waste material but may be opened for draining body waste material from the pouch after a period of use. Such drainable pouches are disclosed, for example, in Nolan, U.S. Pat. No. 3,523,534, and Jensen et al., U.S. Pat. No. 4,411,659, which are incorporated herein in their entirety by reference.

The discharge opening of drainable pouches is typically defined at the end of a narrowed outlet portion, which is provided with closure means for maintaining the discharge opening in a sealed condition until waste material is to be drained from the pouch. The closure means may take the form of a clamp, as in the aforementioned Nolan patent, or a device such as conventional wire ties or wraps for securing the outlet portion in an upwardly-rolled condition.

For quality of life of the users, drainable pouches should be easy to drain without risking soiling of clothes or the surroundings. They also should be easy to close securely after being drained and amenable to being cleaned after drainage and before closing again, such that the risk of unpleasant odor is substantially reduced. Most importantly, the closure means should provide a secure seal when closed to minimize the risk of leakage.

Many different solutions concerning the closing, cleaning and drainage operations have been proposed and implemented. For example, Villefrance et al., U.S. Pat. No. 7,879,015 and Friske et al., U.S. Pat. No. 8,672,907, which are commonly assigned with the present application and incorporated herein in their entirety by reference, disclose drainable pouches having integral closure systems. For obvious reasons, further improvements in the closure systems for easier operation and reduced risk of leakage are much desired by users.

Accordingly, there is a need for an improved closure system for drainable pouches.

### BRIEF SUMMARY

A drainable ostomy pouch comprising an outlet device including a closure member and a slider is provided according to various embodiments. The outlet device may be configured to provide an improved drainable pouch outlet that is easier to handle during draining and cleaning compared to conventional drainable pouch outlets. The outlet device may provide a liquid tight closure via a simple sliding motion using a slider that is configured to engage with a closure member. The outlet device may also be configured to allow a user to open the pouch outlet by applying pressure along opposite sides of the outlet device to provide a discharge opening having angled corners. Further, the outlet device may be formed from a relatively rigid material and provided with bend lines to facilitate opening of the pouch outlet. Such features may eliminate difficulties in opening an ostomy pouch due to the pouch outlet walls sticking together and bending in the same direction. Further, by providing a discharge opening having angled corners, the outlet device may provide improved flow control of body waste draining out through the discharge opening.

In one aspect, a drainable ostomy pouch may include a bodyside wall and an outer wall defining a cavity therebetween for collecting stomal discharge, and a downwardly extending outlet portion terminating in an outlet opening for draining stomal discharge contents collected in the cavity. The drainable ostomy pouch may also include an outlet device comprising a closure member and a slider attached to the outlet portion. The closure member may include a body including a first opening, a second opening, and four bend zones defined therebetween, and at least one bar arranged on the body proximate the second opening. The closure member may be configured to bend along the four bend zones to provide a discharge opening having four angled corners in an open position and to be pressed flat in a closed position. The slider may be configured to slide over the at least one bar and engage with the closure member to close the discharge opening.

The four bend zones may extend longitudinally along the body between the first and second openings. In an embodiment, the body may be formed from a single piece of a polymeric material, in which the bend zones are defined by crease lines or perforated lines. Alternatively, the body may be formed from four pieces of a polymeric sheet material, in which each piece defines a panel of the body, and four boundaries between the panels define the four bend zones.

In some embodiment, the bend zones may be symmetrically arranged, such that a distance between adjacent bend zones is generally equal to each other, and the closure member provides a generally square-shaped discharge opening in a fully opened position. In another embodiment, the bend zones may include first, second, third and fourth bend zones, wherein the first bend zone is adjacent the second and fourth bend zones. A distance between the first and second bend zone may be generally equal to a distance between the third and fourth bend zones, and a distance between the second and third bend zones may be generally equal to a distance between the first and fourth bend zones. In such an embodiment, the distance between the first and second bend zones may be greater than the distance between the second and third bend zones, such that the closure member may provide a generally rectangular discharge opening in a fully opened position.

In an embodiment, the at least one bar may include four bars, in which each of the four bars is arrange on an outer surface of the body proximate the second opening, such that each of the four bend zones is arranged between adjacent bars.

The closure member may include a soft material arranged proximate the second opening. The soft material may be configured and arranged to be compressed together when the closure member engages with the slider to provide a liquid tight seal closure. In an embodiment, the soft material may be attached to inner surfaces of the body opposite the at least one bar. In another embodiment, the soft material may be arranged on outer surfaces of the body between the at least one bar and the body. The soft material may be formed from a foam material.

In some embodiments, the closure member may be arranged in the outlet portion, such that the at least one bar extends beyond the outlet opening, and outer surfaces of the body are attached to adjacent inner surfaces of the outlet portion. The outlet device may be configured and arranged to allow a user to press along sides of the outlet portion to provide the discharge opening having the four angled corners.

In any of the forgoing embodiments, the drainable ostomy pouch may include a two-part fastening system comprising first and second fastener strips configured for securing the outlet portion in a folded up closed position. The outlet portion may be configured to be folded up two times to engage the first and second fastener strips to secure the folded outlet portion, wherein the outlet portion may be configured to fold along an upper periphery of the closure member proximate the first opening for a first fold and fold along a lower periphery of the closure member proximate the second opening for the second fold.

In an embodiment, the slider may have a generally u-shaped body including a channel configured to receive a peripheral portion of the closure member proximate the second opening including the at least one bar. In such an embodiment, the outlet device may be configured to provide a liquid tight seal when the slider engages the closure member and applies pressure over the peripheral portion of the closure member. Further, the slider may be configured to fold up longitudinally and slide over a side periphery of the outlet portion in an open position.

Other aspects, objectives and advantages will become more apparent from the following detailed description when taken in conjunction with the accompanying drawings.

### BRIEF DESCRIPTION OF THE DRAWINGS

The benefits and advantages of the present embodiments will become more readily apparent to those of ordinary skill in the relevant art after reviewing the following detailed description and accompanying drawings, wherein:

FIG. 1 is a perspective view of a drainable ostomy pouch including an outlet device comprising a closure member and a slider according to an embodiment;

FIG. 2 is a perspective view of the drainable ostomy pouch of FIG. 1 in an open position with the slider folded and held in place along a side periphery of a pouch outlet portion;

FIG. 3 is a perspective view of the drainable ostomy pouch of FIGS. 1 and 2 showing the outlet portion fully opened;

FIG. 4 is a perspective view of the outlet device of FIG. 1 including a closure member and a slider according to an embodiment;

FIG. 5 is a perspective view of the closure member of FIG. 4 in an open position;

FIG. 6 is a cross sectional side view of an outlet device including a closure member and a slider according to another embodiment;

FIG. 7 is a perspective view of the drainable pouch of FIG. 1 in a closed position;

FIG. 8 is a perspective view of the drainable pouch of FIG. 7 folded up once;

FIG. 9 is a perspective view of the drainable pouch of FIG. 7 folded up two times and secured in place with a pair of fasteners;

FIG. 10 is a schematic bottom view of a closure member according to an embodiment in a closed position; and

FIG. 11 is a schematic bottom view of the closure member of FIG. 10 in an open position.

### DETAILED DESCRIPTION

While the present disclosure is susceptible of embodiment in various forms, there is shown in the drawings and will hereinafter be described presently preferred embodiments with the understanding that the present disclosure is to be considered an exemplification and is not intended to limit the disclosure to the specific embodiments illustrated. The words “a” or “an” are to be taken to include both the singular and the plural. Conversely, any reference to plural items shall, where appropriate, include the singular.

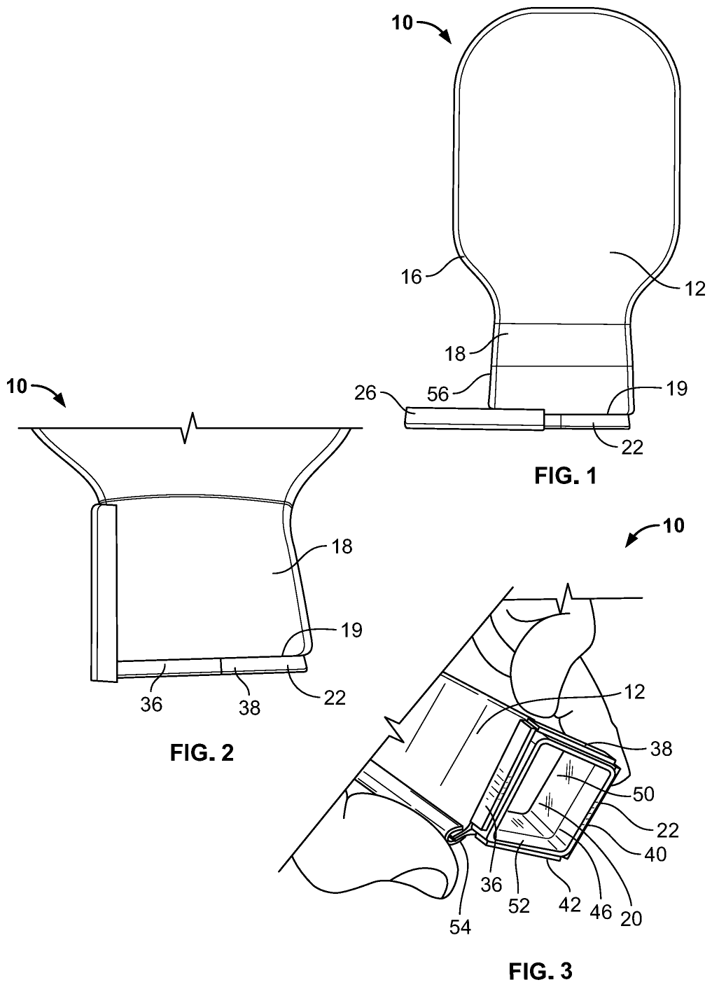

Referring now to the figures, FIGS. 1-3 show a drainable ostomy pouch 10 according to an embodiment. The drainable ostomy pouch 10 may include an outer wall 12 and a body-side wall 14, which may be joined along their peripheral edges 16 to define a cavity therebetween for collecting stomal discharge. The drainable ostomy pouch 10 may include a downwardly extending outlet portion 18 terminating in a discharge opening 20 for draining the contents collected in the cavity after a period of use. An outlet device 22 may be provided in the outlet portion 18 to facilitate opening of the discharge opening 20 and to provide liquid tight closure of the drainable ostomy pouch 10.

The pouch walls 12, 14 may be formed from a suitable flexible material, such as a polymeric film, which may be a monolayer or multilayer film. Each of the pouch walls 12, 14 may be formed of one continuous flexible film to define the entire pouch including the outlet portion 18. Alternatively, the walls of the outlet portion 18 may be formed of separate flexible films than the walls of the pouch body. That is, the walls of the outlet portion 18 may be formed from a different polymeric film than the walls of the pouch body.

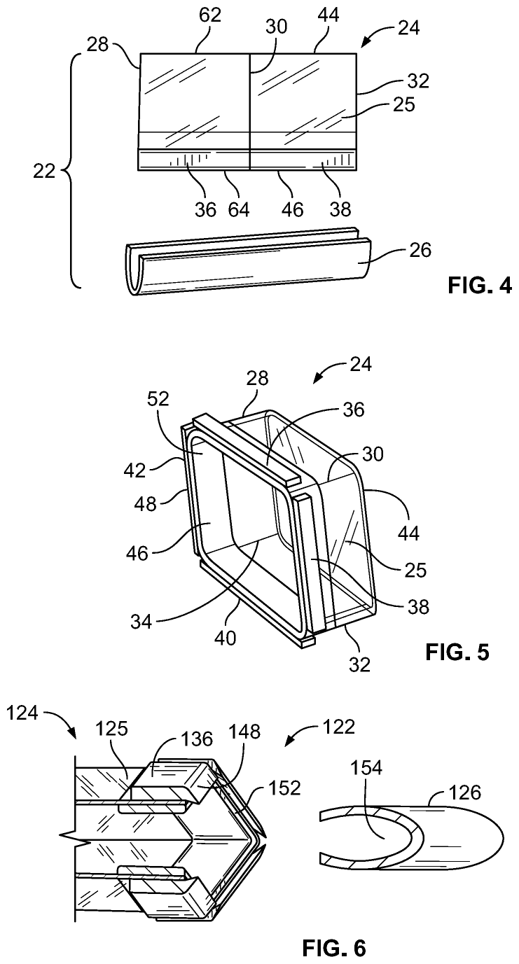

FIGS. 4-5 illustrate an embodiment of the outlet device 22 generally comprising a closure member 24 and a slider 26. The closure member 24 may include a generally rectangular shaped body 25 comprising a first opening 44, a second opening 46, and four bend zones 28, 30, 32, 34 defined therein. The four bend zones 28, 30, 32, 34 may extend longitudinally along the body 25 between the first and second openings 44, 46. The closure member 24 may also include bars 36, 38, 40, 42 arranged on outer surfaces of the body 25 proximate the second opening 46. FIG. 4 shows the closure member 24 in a closed position, in which the closure member 24 is pressed flat. FIG. 5 shows the closure member 24 in an open position, in which the closure member 24 is bent along four bend zones 28, 30, 32, 34 to provide the first and second openings 44, 46 having four angled corners defined at the four bend zones 28, 30, 32, 34.

The body 25 may be formed from a suitable material. In some embodiments, the body 25 may be formed from a relatively rigid polymeric material having a thickness of about 2 mil to about 40 mil. The body 25 may be formed from a single piece of polymeric material, wherein the bend zones 28, 30, 32, 34 may be defined by crease lines or perforated lines. Alternatively, the body 25 may be formed using four separate panels joined together, in which boundaries between the panels define the bend zones 28, 30, 32, 34.

In the embodiments of FIGS. 1-5, the outlet device 22 may include four bars 36, 38, 40, 42. Each of the four bars 36, 38, 40, 42 may be arranged proximate the second opening 46 between bend zones 28, 30, 32 34, such that each of the bend zones 28, 30, 32, 34 is arranged between adjacent bars. In another embodiment, the outlet device 22 may include two bars, each including a bend zone defined therein. In such an embodiment, the two bars may be arranged on opposite outer surfaces of the body 25 proximate the second opening 46, such that two of the bend zones defined in opposite sides of the body 25 are arranged between the two bars and other two bend zones defined in the body 25 line up with the bend zones defined in the bars. In yet another embodiment, the outlet device 22 may include a single bar configured to fit over the body 25 proximate the second opening 46 and including four bend zones defined therein. In such an embodiment, the bar may be arranged, such that the four bend zones defined in the body 25 and the four bend zones defined in the bar may align with each other to allow the outlet device to bend along the bend zones.

The bars 36, 28, 40, 42 may be formed from the same material as the body 25 or a different material. In an embodiment, the bars 36, 38, 40, 42 may be formed from a relatively rigid polymeric material having a greater thickness than the body 25. For example, the bars 36, 38, 40, 42 may be formed from a polymeric material having a thickness of about 5 mil to about 80 mil. In one embodiment, the closure member 24 including the body 25 and the bars 36, 38, 40, 42 may be formed via injection molding.

In an embodiment, the bend zones 28, 30, 32, 34 may be symmetrically arranged, such that a distance between adjacent bend zones is generally equal to each other. In such an embodiment, the closure member 24 may provide a generally square-shaped discharge opening 20 when fully opened as shown in FIG. 3. Each of the bars 36, 38, 40, 42 may be configured to have a generally rectangular shape with generally same width, length, and thickness. The bars 36, 38, 40, 42 may be provided in the areas between the four bend zones 28, 30, 32, 34 of the body 25 proximate the second opening 46, as shown in FIGS. 3 and 5.

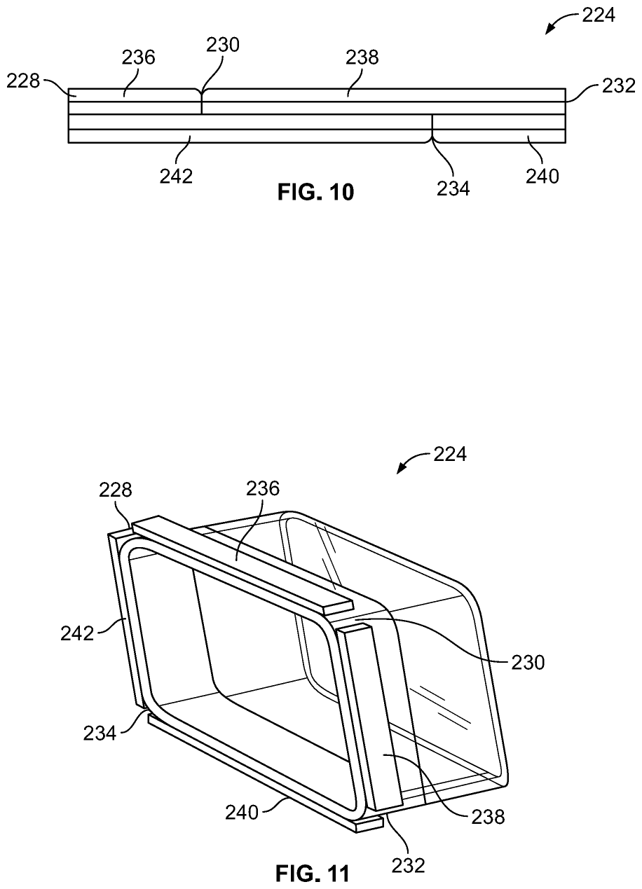

Alternatively, a closure member may be configured such that distances between adjacent bend zones are not all equal to each other. FIGS. 10-11 are schematic bottom views of a closure member 224 having such configuration of bend zones according to an embodiment. The closure member 224 may be configured similar to the closure member 24 of FIGS. 1-5 including a generally rectangular shaped body comprising a first opening, a second opening 246, four bend zones 228, 230, 232, 234, and four bars 236, 238, 240, 242. In this embodiment, a distance between the bend zones 228 and 230 is generally equal to a distance between the bend zones 232 and 234, and greater than a distance between the bend zones 230, 232 and a distance between the bend zones 228, 234. The distance between the bend zones 230, 232 and the distance between the bend zones 228, 234 are generally equal to each other.

As such, two longer bars 236, 240 and two shorter bars 238, 242 may be arranged between the bend zones 228, 230, 232, 234 as shown in FIG. 10. When opened, the closure member 224 may provide a generally rectangular discharge opening as shown in FIG. 11. The closure member 224 having such a configuration may be less likely to fold in on itself when compared to a closure member having bend zones arranged generally an equal distance from each other, such as the closure member 24 of FIGS. 1-5, to allow the closure member 224 to remain in an open position.

In an embodiment, each of the bars 36, 38, 40, 42 may be provided with a sharp edge 48 along a peripheral edge. The sharp edges 48 may be defined by a reduced thickness portion of the bars 36, 38, 40, 42 or a separate member having a sharper edge than the edge of the bars 36, 38, 40, 42. In such an embodiment, each of the bars 36, 38, 40, 42 may be arranged on the outer surfaces of the body 25 proximate the second opening 46, such that the sharp edges 48 may extend beyond an outer periphery of the body 25. The sharp edges 48 may be configured to reduce or prevent dripping and/or spilling when emptying body waste collected in an ostomy pouch through the discharge opening 20. In some embodiments, the bars 36, 38, 40, 42 may be configured to facilitate attachment of an adaptor for a night drainage bag.

The closure member 24 may also include a soft material 52 proximate the second opening 46 to facilitate sealing of the drainable ostomy pouch 10 in a closed position. In an embodiment, the soft material 52 may be attached to inner surfaces of the body 25 opposite the bars 36, 38, 40, 42 proximate the second opening 46 as shown in FIGS. 3 and 5. In another embodiment, the soft material may be arranged on outer surfaces of the body 25 between the bars 36, 38, 40, 42 and the body 25. The soft material 52 may be formed from a suitable soft material, such as a foam material, soft silicone, nitrile rubber or similar materials known for good sealing properties. For example, the soft material 52 may be formed from a closed cell foam material and arranged on the inner surfaces of the body 25 opposite the bars 36, 38, 40, 42. The soft material 52 may be configured and arranged to be compressed together when the closure member 24 engages with the slider 26 to provide a liquid tight seal closure.

FIG. 6 is a cross sectional side view of an outlet device 122 according to another embodiment. The outlet device 122 may be configured similar to the outlet device 22 and generally include a closure member 124 and a slider 126. The closure member 124 may include a body 125, bars 136, and a soft material 152. In this embodiment, each of the bars 136 may be provided with a protruding end portion 148 forming a sharp edge. The protruding end portions 148 may be formed from a suitable thin material having a thickness of about 2 mil to about 10 mil to provide the sharp edge extending from the bars 136.

The slider 26 may be configured to engage with the closure member 24 to provide a liquid tight closure for the ostomy pouch 10. In an embodiment, the slider 26 may be formed from a suitable polymeric material and configured to slide over the bars 36, 38, 40, 42 as shown in FIG. 1. In the embodiments of FIGS. 1-4 and 6, the slider 26, 126, may have a generally u-shaped body including a channel 54, 154 configured to receive a peripheral portion of the closure member 24, 124 including the bars 36, 38, 40, 42, 136. The outlet device 22, 124 may be configured to provide a liquid tight seal when the slider 26, 126 engages the closure member 24, 124 and applies pressure over the peripheral portion of the closure member 24, 124.

In an embodiment, the slider 26 may be configured to fold up longitudinally and slide over a side periphery 56 of the pouch outlet portion 18 as shown in FIGS. 2 and 3. In such an embodiment, the outlet device 22 may be provided with a stop (not shown) proximate a corner of the outlet device configured to prevent the slider 26 from sliding completed off the closure member 24. In an open position, the slider 26 may be folded up longitudinally and held in place along the side periphery 56 as shown in FIG. 2, which can prevent the slider 26 from being misplaced during draining and cleaning.

In an embodiment, the closure member 24 may be arranged in the outlet portion 18 of the drainable pouch 10, such that the bars 36, 38, 40, 42 extend beyond an outer periphery 19 of the outlet portion 18 as shown in FIGS. 1-3. In such an embodiment, outer surfaces of the body 25 may be attached to adjacent inner surfaces of the outlet portion 18 via heat sealing, an adhesive, or other similar known methods. In another embodiment, the closure member 24 may be arranged over the outlet portion 18, such that the inner surfaces of the body 25 are attached to the adjacent outer surfaces of the outlet portion 18.

When attached to the outlet portion 18, the outlet device 22 may allow a user to press along sides of the outlet portion 18 to provide the discharge opening 20 having angled corners defined at the four bend zones 28, 30, 32, 34. In some embodiments, the outlet device 22 may provide generally v-shaped corners in an open position as shown in FIGS. 3 and 5. Further, the outlet device may provide a generally square shaped discharge opening 20 in a fully opened position as shown in FIG. 3. An outlet path 50 is defined in the closure member 24 between the first and second opening 44, 46 providing a flow path for draining body waste collected in the drainable pouch 10.

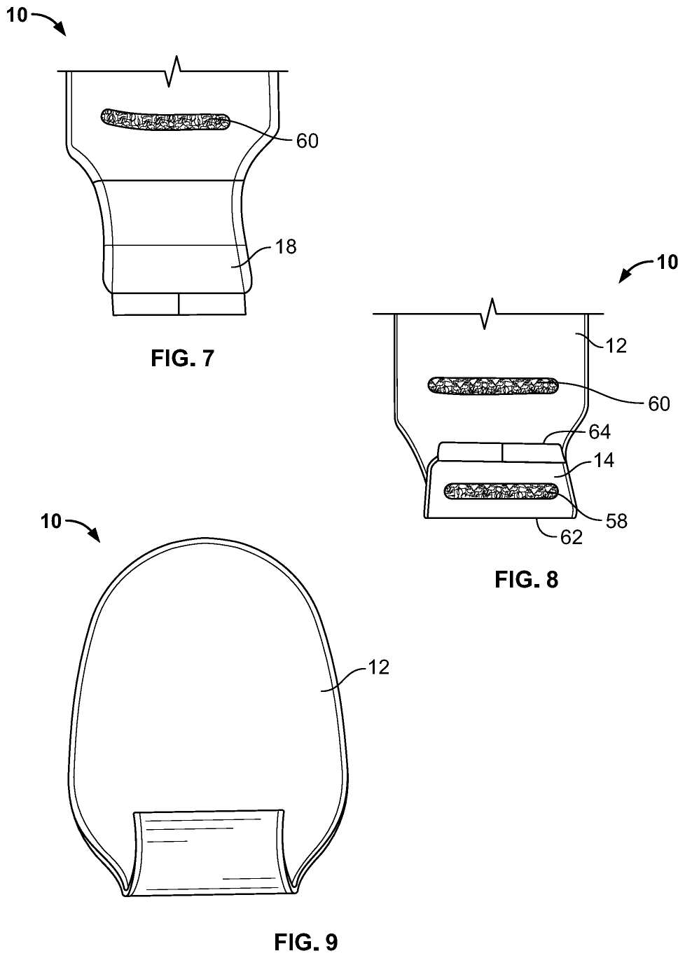

In an embodiment, the drainable pouch 10 may include a two-part fastening system comprising first and second fastener strips 58, 60 for securing the folded-up outlet portion 18 in a closed position as shown in FIGS. 7-9. The first fastener strip 58 may be provided on an outer surface of the body-side wall 14 in the outlet portion 18, while the second fastener strip 60 may be arranged on an outer surface of the outer wall 12 in a pouch portion as shown in FIGS. 7-8. The first and second fastener strips 58, 60 may include fastener elements, which are adapted for releasable interlocking engagement. In the embodiments of FIGS. 7-9, the drainable pouch 10 may be configured such that the outlet portion 18 may be folded up two times to engage the first and second fastener strips 58, 60 to secure the folded outlet portion 18. In this embodiment, the outlet portion 18 may be configured to fold along an upper periphery 62 of the closure member 24 (FIGS. 4 and 8) for a first fold and fold along a lower periphery 64 of the closure member 24 (FIGS. 8 and 9) for a second fold. The fastener strips 58, 60 may be formed from known fastening means, such as, for example, hook and loop fasteners as marketed under the Velcro trademark or pressure sensitive adhesive coatings.

From the foregoing it will be observed that numerous modifications and variations can be effectuated without departing from the true spirit and scope of the novel concepts of the present disclosure. It is to be understood that no limitation with respect to the specific embodiments illustrated is intended or should be inferred. The disclosure is intended to cover by the appended claims all such modifications as fall within the scope of the claims.

1. A drainable ostomy pouch, comprising:
a bodyside wall and an outer wall defining a cavity therebetween for collecting stomal discharge;
a downwardly extending outlet portion terminating in an outlet opening for draining stomal discharge contents collected in the cavity; and
an outlet device attached to the outlet portion, wherein the outlet device comprises a closure member and a slider, the closure member comprising:
a body including first opening, second opening, and four bend zones defined therebetween; and
at least one bar arranged on the body proximate the second opening;
wherein the closure member is configured to bend along the four bend zones to provide a discharge opening having four angled corners in an open position and to be pressed flat in a closed position;
wherein the slider is configured slide over the at least one bar and engage with the closure member to close the discharge opening.

2. The drainable ostomy pouch of claim 1, wherein the four bend zones extend longitudinally along the body between the first and second openings.

3. The drainable ostomy pouch of claim 1, wherein the body is formed from a single piece of a polymeric material, wherein the bend zones are defined by crease lines or perforated lines.

4. The drainable ostomy pouch of claim 1, wherein the body is formed from four pieces of a polymeric sheet material, wherein each piece defines a panel of the body, wherein four boundaries between the panels define the four bend zones.

5. The drainable ostomy pouch of claim 1, wherein the bend zones are symmetrically arranged, wherein a distance between adjacent bend zones is generally equal to each other, and wherein the closure member provides a generally square-shaped discharge opening in a fully opened position.

6. The drainable ostomy pouch of claim 1, wherein the bend zones include first, second, third and fourth bend zones, wherein the first bend zone is adjacent the second and fourth bend zones, wherein a distance between the first and second bend zone is generally equal to a distance between the third and fourth bend zones, and a distance between the second and third bend zones is generally equal to a distance between the first and fourth bend zones, wherein the distance between the first and second bend zones is greater than the distance between the second and third bend zones, and wherein the closure member provides a generally rectangular discharge opening in a fully opened position.

7. The drainable ostomy pouch of claim 1, wherein the at least one bar includes four bars, wherein each of the four bars is arrange on an outer surface of the body proximate the second opening, such that each of the four bend zones is arranged between adjacent bars.

8. The drainable ostomy pouch of claim 1, wherein the closure member includes a soft material arranged proximate the second opening, wherein the soft material is configured and arranged to be compressed together when the closure member engages with the slider to provide a liquid tight seal closure.

9. The drainable ostomy pouch of claim 8, wherein the soft material is attached to inner surfaces of the body opposite the at least one bar.

10. The drainable ostomy pouch of claim 8, wherein the soft material is arranged on outer surfaces of the body between the at least one bar and the body.

11. The drainable ostomy pouch of claim 1, wherein the soft material is formed from a foam material.

12. The drainable ostomy pouch of claim 1, wherein the closure member is arranged in the outlet portion, such that the at least one bar extends beyond the outlet opening, wherein outer surfaces of the body are attached to adjacent inner surfaces of the outlet portion, wherein the outlet device is configured and arranged to allow a user to press along sides of the outlet portion to provide the discharge opening having the four angled corners.

13. The drainable ostomy pouch of claim 1, wherein the drainable ostomy pouch includes a two-part fastening system comprising first and second fastener strips configured for securing the outlet portion in a folded up closed position.

14. The drainable ostomy pouch of claim 13, wherein the outlet portion is configured to be folded up two times to engage the first and second fastener strips to secure the folded outlet portion, wherein the outlet portion is configured to fold along an upper periphery of the closure member proximate the first opening for a first fold and fold along a lower periphery of the closure member proximate the second opening for the second fold.

15. The drainable ostomy pouch of claim 1, wherein the slider has a generally u-shaped body including a channel configured to receive a peripheral portion of the closure member proximate the second opening including the at least one bar, wherein the outlet device is configured to provide a liquid tight seal when the slider engages the closure member and applies pressure over the peripheral portion of the closure member.

16. The drainable ostomy pouch of claim 1, wherein the slider is configured to fold up longitudinally and slide over a side periphery of the outlet portion in an open position.
